(19) United States
(12) Patent Application Publication
(10) Pub. No.: US 2012/0150003 A1
(43) Pub. Date: Jun. 14, 2012

(54) System Non-invasive Cardiac Output Determination

(75) Inventor: Hongxuan Zhang, Palatine, IL (US)

(73) Assignee: SIEMENS MEDICAL SOLUTIONS USA, INC., Malvern, PA (US)

(21) Appl. No.: 13/215,307

(22) Filed: Aug. 23, 2011

Related U.S. Application Data
(60) Provisional application No. 61/421,234, filed on Dec. 9, 2010.

(51) Int. Cl. A61B 5/0205; A61B 5/1455

(52) U.S. Cl. 600/324; 600/323

(57) ABSTRACT

A system determines cardiac output and stroke volume by using non-invasive oximetric signals, such as SPO2 data and waveform, to determine blood flow quantitatively. A non-invasive system determines cardiac output or stroke volume. The system includes an input processor for receiving signal data representing oxygen content of blood of a patient at a particular anatomical location. A computation processor uses the received signal data in calculating a heart stroke volume of the patient comprising volume of blood transferred through the blood vessel in a heart cycle, in response to, a blood volume derived in response to oxygen content of patient blood and at least one factor representing reduction in blood flow volume from a patient heart to the particular anatomical location. An output processor provides data representing the calculated heart stroke volume to a destination device.

This is a non-provisional application of provisional application Ser. No. 61/421,234 filed 9 Dec. 2010, by H. Zhang.

### FIELD OF THE INVENTION

This invention concerns a system for determining cardiac output and stroke volume in response to, a blood volume derived in response to oxygen content of patient blood and at least one factor representing reduction in blood flow volume from a patient heart to a particular anatomical location.

### BACKGROUND OF THE INVENTION

Cardiac output (CO) or stroke volume (SV) involve measurements of blood volume ejected by a left ventricle in one minute or in one heart beat and are valuable vital sign signals used for patient health status monitoring. There are multiple methods to calculate CO and SV including using a blood pressure waveform, thermodilution, bio-impedance, a pulse contour or ultrasound, for example. However most of these clinical methods are invasive and unreliable which limits their use and results in additional risk to patients. Accurate clinical assessment of patient circulatory status is desirable especially in critically ill patients in an ICU (intensive care unit) and patients undergoing cardiac, thoracic, or vascular interventions. As patient hemodynamic status may change rapidly, continuous monitoring of cardiac output provides information allowing rapid adjustment of therapy. CO and SV are valuable parameters used for cardiac function evaluation and associated calculations. Known methods for CO and SV determination include indicator dilution methods, Fick principle methods, Bio-impedance and conduction methods, Doppler ultrasound methods and arterial pulse contour analysis methods. However these methods have different limitations and disadvantages

Known clinical methods for CO and SV calculation are mostly invasive and require catheters and this adds to clinical procedure complexity and poses additional risk to patients. The known clinical methods for CO, SV calculation require extensive clinical experience and knowledge for interpretation of the parameters and for calculation accuracy and are also often complex, and time consuming and may be unsuitable for particular clinical environments. Further known cardiac output calculation methods may be dependent on sensor quality and be sensitive to noise (such as from a power line, patient movement, or treatment, such as pacing and drug delivery) resulting in an unreliable cardiac function calculation. A system according to invention principles addresses these deficiencies and related problems.

### SUMMARY OF THE INVENTION

A system determines cardiac output and stroke volume by using non-invasive oximetric signals, such as SPO2 data and associated waveform, to determine blood flow quantitatively. A non-invasive system determines cardiac output or stroke volume. The system includes an input processor for receiving signal data representing oxygen content of blood of a patient at a particular anatomical location. A computation processor uses the received signal data in calculating a heart stroke volume of the patient comprising volume of blood transferred through the blood vessel in a heart cycle, in response to, a blood volume derived in response to oxygen content of patient blood and at least one factor representing reduction in blood flow volume from a patient heart to the particular anatomical location. An output processor provides data representing the calculated heart stroke volume to a destination device.

### BRIEF DESCRIPTION OF THE DRAWING

FIG. 1 shows a non-invasive system for determining cardiac output or stroke volume, according to invention principles.

FIG. 2 shows determination of blood flow from heart to body capillaries, such as in a finger tip using measured SPO2 oximetric parameters, according to invention principles.

FIG. 3 illustrates continuously acquired SPO2 data, according to invention principles.

FIG. 4 shows an artificial neural network (ANN) for time varying and nonlinear blood flow calculation, according to invention principles.

FIG. 5 shows a flowchart of a process used for determining cardiac output and stroke volume using SPO2 oximetric signals, according to invention principles.

FIG. 6 shows SPO2 signal based CO and SV calculation during normal rest and exercise episodes of a patient, according to invention principles.

FIG. 7 shows a flowchart of a process used by a non-invasive system for determining cardiac output or stroke volume, according to invention principles.

### DETAILED DESCRIPTION OF THE INVENTION

A system determines cardiac output and stroke volume by using non-invasive oximetric signals, such as blood oxygen saturation (SPO2) data to quantitatively determine blood flow. The SPO2 data is utilized to analyze heart function and blood flow characteristics by building a bridging model between non-invasive blood oximetric signals in capillaries (such as in a finger tip) and cardiac pumps comprising heart chambers (particularly a left ventricle). Using nonlinear modeling based on SPO2 signal properties (such as Density, Variability, Variation), the system accurately determines cardiac output in the presence or absence of substantial noise. The system detects cardiac disorders, differentiates between cardiac arrhythmias, characterizes pathological severity, predicts life-threatening events, and facilitates evaluation of the effects of drug administration to a patient.

The system quantitatively determines CO and SV values by determining a blood oxygen content (SPO2) representative parameter. Typically SPO2 is typically used to measure blood oxygen content in capillaries, for example, to determine patient health status, such as asthma severity and identify atrial fibrillation. SPO2 data is also used for other applications, such as blood flow estimation and hemodynamic parameter estimation. The system uses SPO2 (oximetry data) to calculate cardiac output and stroke volume. The system advantageously derives and uses a relationship between SPO2 oximetric signal measurements and heart cardiac output. SPO2 is a vital sign used to monitor and diagnose patient health status, by measuring the saturation of hemoglobin with oxygen as measured by pulse Oximetry, for example. The link between heart pump (CO) activity and blood flow in small blood vessels (capillaries) is advantageously derived herein. SPO2 data may be acquired by non-invasive sensors using infrared light, such as by using known SPO2 acquisition sensor systems. Usually these sensor systems (including OEM devices) output a continuous data stream derived using a sample rate from 20-100 Hz, for example. The system uses the digitized data output to calculate SPO2 characteristics and parameters, such as density, energy and dynamic variation and variability.

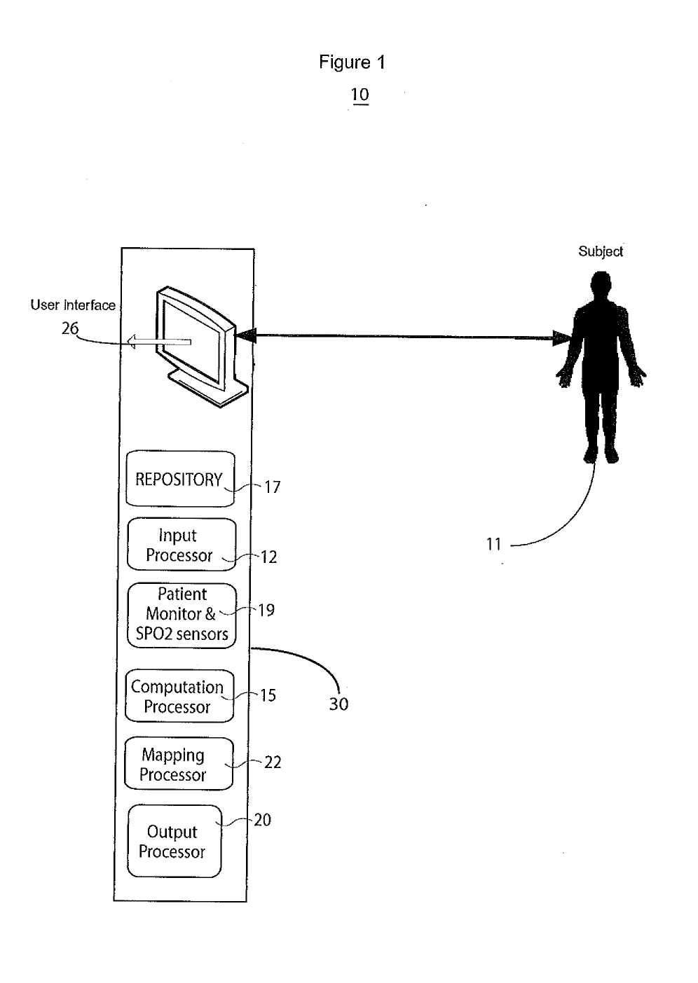

FIG. 1 shows system 10 for heart performance characterization and abnormality detection. System 10 comprises at least one computer system, workstation, server or other processing device 30 including input processor 12, repository 17, mapping processor 22, patient monitoring devices and SPO2 measurement sensor 19, computation processor 15, output processor 20 and a user interface 26. Input processor 12 receives signal data representing oxygen content of blood of patient 11 at a particular anatomical location derived by blood oxygen content (SPO2) measurement sensor 19. Computation processor 15 uses the received signal data in calculating a heart stroke volume of patient 11 comprising volume of blood transferred through the blood vessel in a heart cycle, in response to, a blood volume derived in response to oxygen content of patient blood and at least one factor representing reduction in blood flow volume from a patient heart to the particular anatomical location. Output processor 20 provides data representing the calculated heart stroke volume to a destination device. Blood containing oxygen flows to a left ventricle and is pumped out by the left ventricle to the main artery which transports oxygenated blood to the body, from vessel to organ, from big vessel to small vessel and to capillaries. Patient monitoring devices and SPO2 measurement sensor 19 acquires non-invasive SPO2 oximetric signals using light sensors located on or near capillaries of patient 11.

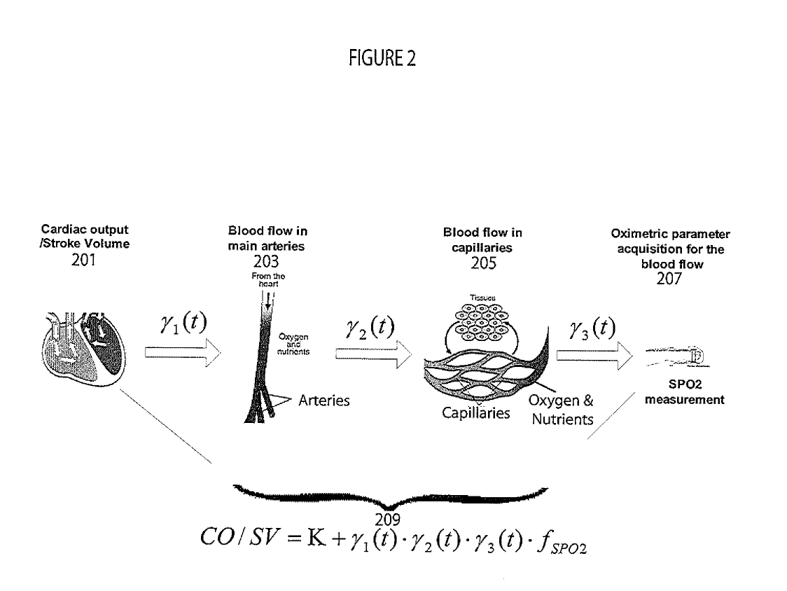

FIG. 2 shows determination of blood flow from heart to body capillaries, such as in a finger tip using SPO2 oximetric parameters measured by sensors 19 (FIG. 1). The Figure shows blood flow from heart to capillaries and associated linear and nonlinear ratios in the flow sequence. Typically a left ventricle pumps blood 201 into main arteries 203 which transport the blood to small blood vessels and organs, and eventually to body capillaries 205. In each step, the blood volume is proportionally reduced such as by a ratio γ₁(t) representing degree of transition from a heart. Based on timing and blood vessel volume, the ratio γ₁(t) may be time varying and nonlinear. ƒ_{SPO2} is a function used to calculate blood flow and volume from SPO2 data 207. Computation processor 15 (FIG. 1) determines the cardiac output and stroke volume using,

CO/SV=K+β₁(t)·γ₂(t)·γ₃(t)·ƒ_{SPO2}

where γ₁(t), γ₂(t), and γ₃(t) are volume ratios in each stage of FIG. 2 indicating blood flow volume reduction, K represents a baseline and static portion of blood flow and volume. It is known CO=Heart rate×SV and, CO and SV comprise a cardiac output calculation. Further, ƒ_{SPO2} is calculated as a function of multiple parameters as,

ƒ_{SPO2}=ƒ(Density,max,min,mean,std,variablility,variation,HOS)

where max is the maximum value of the SPO2 data in a time period, min is the minimum value of the SPO2 data in the time period, mean is the average value of the SPO2 data in the time period; std is the standard deviation of the SPO2 value in the time period; variability is a statistical parameter for the SPO2 value in the time period determined as described later. The std and variability are computed for a data stream of SPO2 data comprising a dataset in the time period used for max, min, mean, determination. HOS means high order statistical calculated value, such as a bi-spectrum value. In calculating ƒ_{SPO2}, one or more of, and less than all the parameters, density, max, min, std, variability, variation and HOS may be used to calculate ƒ_{SPO2} but the sensitivity and accuracy may be improved if more factors and parameters are used in the calculation. Density represents an SPO2 waveform calculated parameter derived using for example one of the following,

$$SPO 2\_Density = \frac{1}{N} {\int }_{i ∈ N} \vert {data}_{i} \vert d t ( Amplitude )$$

$$OR SPO 2\_Density = \frac{1}{N} {\int }_{i ∈ N} {\vert {data}_{i} \vert}^{2} d t ( Energy )$$

where N is the number of data samples in the density calculation window and data is an SPO2 data value in an SPO2 waveform. For example, there are 6 samples in a one-cycle SPO2 data set: 0.56, 0.75, 1, 0.91, 0.64, 0.55 (these are data values normalized by comparison with a maximum value in the SPO2 waveform), N is 6 and corresponding amplitude SPO2_Density is 0.74 and energy SPO2_Density is 0.57.

Processor 15 calculates mean, standard deviation variation and variability as follows.

$$Mean or average value ( expectation ) ;$$

$$mean ( X ) = \frac{1}{M} {\sum }_{i ∈ M} X ( i ) ;$$

$$Standard deviation \text{:} STD ( X ) = \frac{1}{M - 1} {\sum }_{i ∈ M - 1} ( X ( i ) - mean ( X ) )$$

$$Signal Variation = \frac{mean ( X )}{STD ( X )}$$

$$Signal Variability = \frac{max ( X - mean ( X ) )}{mean ( X )}$$

where X comprises a data series of SPO2 data stream samples, an SPO2 maximum data value series, an SPO2_Density data series or another SPO2 signal data series or derived calculated value series. In the equation, M is a number of data values in a data set in a calculation. The statistical calculation and computation window is 5 to 20 heart beats which also means 5-20 cycles for an SPO2 waveform.

Parameters γ₁(t), γ₂(t), γ₃(t), γ(t), K and λ(t) are different factors, coefficients and ratios in the CO and SV calculation based on SPO2 signal data. K represents a baseline and static portion of blood flow and volume which does change due to patient exercise or time in a cardiac output calculation and K is dependent on patient demographic data, such as weight, skin area and height. Also γ₁(t), γ₂(t), γ₃(t), γ(t) are factors representing cardiac output and blood flow reduction from ventricle to vessel and to capillaries. Parameter λ(t) is a factor associating blood flow volume and oxygen content in a capillary. These factors and coefficients are stable if patient status is stable. However, factors Mt), γ₁(t), γ₂(t), γ₃(t), γ(t), K and λ(t) may change and be time-varying due to patient status and activity including, exercise, cardiac arrhythmia and administration of medication. In the CO and SV determination, these factors are adaptively and automatically controlled and adjusted by a user or by system 10 (FIG. 1) in response to patient status. System 10 or a user adaptively adjusts these coefficients in response to indicators, such as heart rate, respiration rate, patient temperature, and other patient body and vital sign signals.

The blood flow in capillaries is calculated using SPO2 oximetric values via the function ƒ_{SPO2}. In response to data indicating a type of clinical application or procedure being performed (e.g. monitoring for atrial fibrillation, or another heart condition) and user data input, processor 15 derives a function between blood volume flowing in a capillary and SPO2 oximetric data to determine ƒ_{SPO2} a function used to calculate blood flow and volume from SPO2 data. For example, the function uses SPO2 waveform density, max, min, average and variation in,

$${f}_{SPO 2} = \frac{SPO 2\_Density}{λ ( t )} \cdot \frac{Mean ( max ) - Mean ( min )}{Variation ( SPO 2\_average )}$$

where, Mean(max) is a mean of the maximum values of the SPO2 data (here there are N SPO2 heart cycles, similar to ECG signal heart beat cycles), Mean(min) is a mean of the minimum values of the SPO2 data set, Variation(SPO2_average) is a variation parameter derived from an SPO2 average value data set, λ(t) is a ratio between blood flow volume in a capillary and oxygen content, usually 0<λ(t)<1 and λ(t) may be time varying. In a noisy environment, more calculation parameters may be utilized in the ƒ_{SPO2} calculation, including HOS and variability parameters, for example, as previously described. Processor 15 performs a time varying analysis based on patient status including respiration status and pathology. A time varying analysis uses an intelligent lookup table and adaptive process for CO and SV determination.

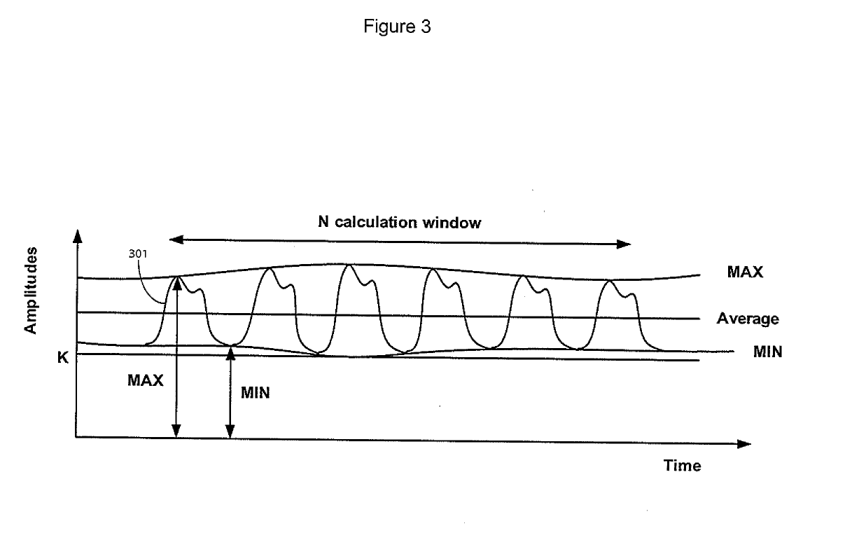

FIG. 3 illustrates continuously acquired SPO2 data 301 indicating parameters K and N as well as max value, average value and min value of an SPO2 dataset. N is calculation window size (e.g., here N=6 cycles). Computation processor 15 (FIG. 1) analyzes the SPO2 waveform to derive SPO2 oximetric information including max, min, and density values. In the CO and SV calculation, parameters used include, SPO2 waveform and data set parameters including max, min, average, std (standard deviation), variability, variation, N (number of heart cycles), time varying factors and ratios, such as γ₁(t), γ₂(t), γ₃(t), and patient factors (e.g., K). Processor 15 calculates characteristic SPO2 dataset parameters. The time varying factors and blood flow associated ratios, such as from heart to artery, from artery to capillaries, are not derived by the system directly since these ratios may be time varying and nonlinear and depend on clinical environment and patient status, such as heart rate and occurrence of arrhythmia. The patient factors comprise patient weight, pathology (such as asthma), patient skin surface area, age, gender, drug delivery and treatment. These kinds of factors and variables are taken into account using parameter K. Hence sometime, K is also varied based on patient status and is represented as K(patient). However K(patient) is stable for one specific patient and may be a small factor such that K(patient)=μK, where μ is usually between 0.95 to 1.05. Thereby the CO calculation comprises,

CO/SV=μK+γ₁(t)·γ₂(t)·γ₃(t)·ƒ_{SPO2}

Or CO/SV=μK+γ(t)·ƒ_{SPO2}

where γ(t) is an overall ratio and factor for blood flow reduction.

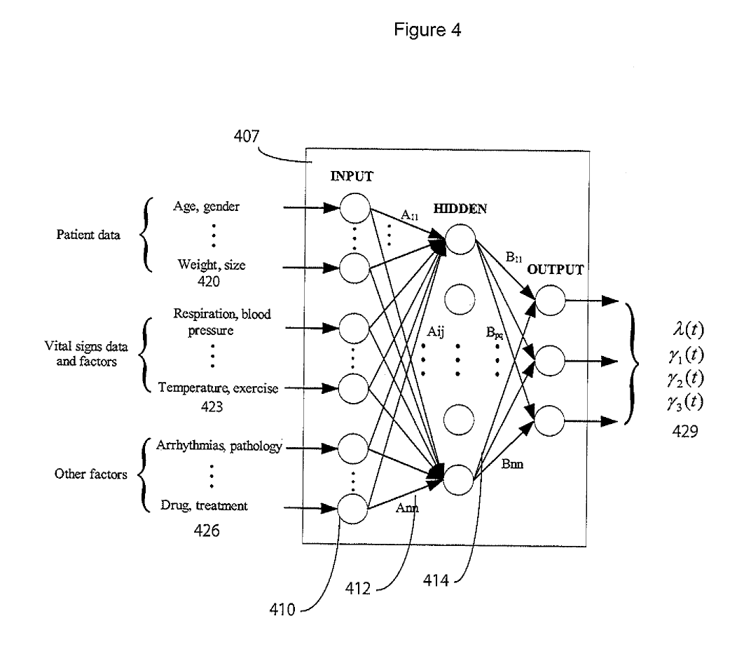

FIG. 4 shows an artificial neural network (ANN) for time varying and nonlinear blood flow calculation and determination of time varying factors, γ₁(t), γ₂(t), γ₃(t) or γ(t). System 10 (FIG. 1) may employ different methods in factor determination, such as Fuzzy modeling or an expert system. ANN unit 407 is used to estimate overall time varying and nonlinear factor γ₁(t), γ₂(t), γ₃(t) and/or γ(t), λ(t).

ANN unit 407 integrates and nonlinearly combines multiple kinds of patient information since different types of patient data and data patterns may have a nonlinear relationship. ANN unit 407 comprises a three layer architecture for combining and integrating different kinds of blood pressure measurements, demographic signals, vital signs and ECG signals, for example. ANN unit 207 combines or maps patient data 420 (including age, weight height, gender), patient parameter and status data 423 (including respiration, blood pressure, temperature, data values and patient activity status) and patient medical condition data 426 (including arrhythmia, pathology, medication), to output parameter γ₁ (t), γ₂(t), γ₃(t) or γ(t) 429. Measurements and calculations are combined nonlinearly to derive a severity indicator and pathology indicator. The indicators are used for statistical tests and validation to identify a dynamic statistical pattern for blood pressure signal pattern quantification and patient cardiac arrhythmia characterization.

ANN unit 407 structure comprises 3 layers, an input layer 410, hidden layer 412 and output layer 414. ANN unit Aᵢⱼ weights are applied between input layer 410 and hidden layer 412 components of the ANN computation and B^{pq} weights are applied between hidden layer 412 and calculation components 414 of the ANN computation. The Aᵢⱼ weights and B^{pq} weights are adaptively adjusted and tuned using a training data set. ANN unit 407 incorporates a self-learning function that processes signals 420, 423 and 426 to increase the accuracy of calculated results. Following a training phase with a training data set, ANN unit 407 maps signals 420, 423 and 426 to data 429. Different types of signal measurements and derived parameters in one embodiment are used independently to determine patient status based on blood pressure cycle interval reflecting cardiac reperfusion rate, a blood pressure waveform integration parameter indicating stroke volume and blood pressure waveform morphology statistics indicating blood perfusion and contraction regularity.

ANN unit 407 (and data processor 15) in one embodiment calculates nonlinear signal parameter,

$$index\_i = {\sum }_{j ∈ Ω} {α}_{ij} ( t ) \cdot {C}_{j}$$

where index_i is an output index from ANN unit 407 representing pathology severity, location and timing, Cⱼ represents a parameter derived from the blood pressure signals, other calculations, and other inputs of the ANN unit, α_{if} (t) represents weights and coefficients. Cⱼ and αᵢⱼ(t) may be adaptively selected in response to procedure type and patient medical condition indicator. In ANN unit 407, αᵢⱼ (t) may be derived in response to a training data set, Ω represents the inputs, including direct patient signal measurements, calculated index, user input and patient demographic data. In a clinical application, different indices may be named according to the meaning and application purpose, such as pathology severity index_{—}1, arrhythmia location index index_{—}2, probability of arrhythmia occurrence index_{—}3, arrhythmia type index_{—}4, EOS (end-of systole) phase interval index_{—}5, blood pressure cycle index_{—}6, domain frequency value index_{—}7 and warning and treatment priority index_{—}8. A dynamic signal pattern indicator is calculated from multiple parameters to indicate a statistical probability and level of patient pathology, event timing, drug delivery effects, to predict a malfunction trend and potential clinical treatment.

In different clinical procedures and different heart rhythms, an index typically shows different values and distribution (indicated by mean value and standard deviation). The system determines a sequential calculation value indicating severity, type, timing and priority, for example. Unit 407 (or processor 15) employs a shifting window (determined by unit 407 or 15 adaptively and automatically in response to sensitivity and noise within data) for processing a sequential index data series for index_{—}1, S1, for example. A ten data point window is used n one embodiment. For each window, a mean value mean(S1), standard deviation STD(S1), variation and variability are calculated using,

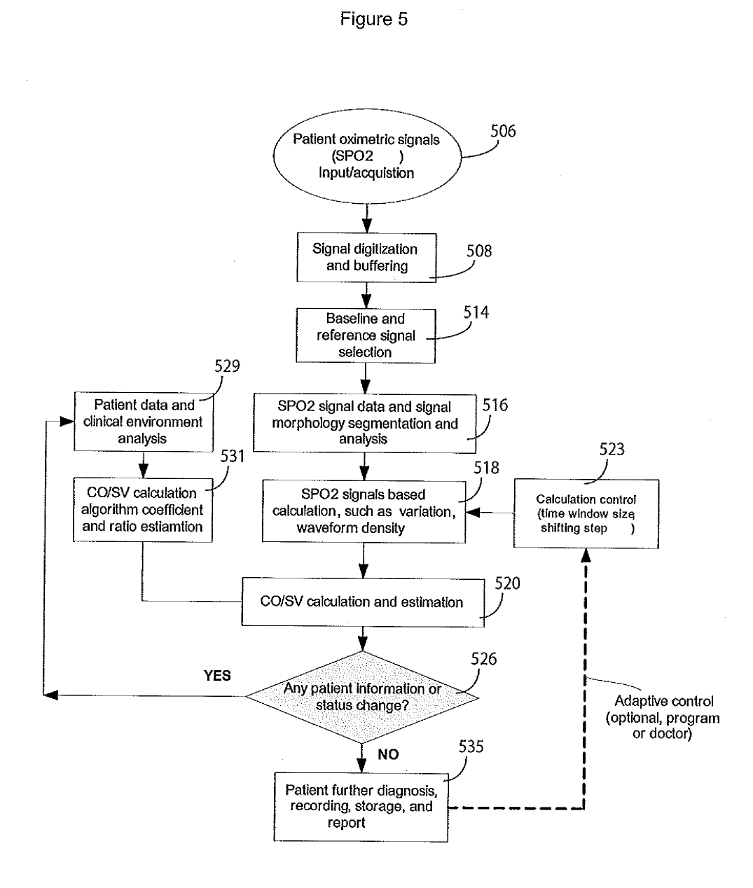

FIG. 5 shows a flowchart of a process used by system 10 (FIG. 1) for determining cardiac output and stroke volume using SPO2 oximetric signals. Input processor 12 in step 508 processes signal data representing oxygen content of blood of a patient acquired from SPO2 sensors 19 at a particular anatomical location by buffering and digitizing the signal data received in step 806. Input processor 12 filters the received signal data using a filter for attenuating power line noise, respiration and patient movement noise and acquires patient information such as weight, age, gender. In step 514, computation processor 15 determines a baseline of the signal data in a detected SPO2 oximetric cycle. Computation processor 15 in step 516 identifies different segments of the filtered signal data and analyzes the signal data to identify signal maximum and minimum values and analyzes the determined patient baseline data for use in CO and SV calculation. Processor 15 uses a peak detector and time detector for identifying the peaks and wave segments and detects peaks within received signal data using a known peak detector and by segmenting the signal into windows where waves are expected and identifying the peaks within the windows. The start point of a wave, for example, is identified by a variety of known different methods. In one method a wave start point comprises where the signal crosses a baseline of the signal (in a predetermined wave window, for example). Alternatively, a wave start point may comprise a peak or valley of signal. The baseline of the signal may comprise a zero voltage line if a static (DC) voltage signal component is filtered out from the signal. Processor 15 includes a timing detector for determining time duration between the signal peaks and valleys. The time detector uses a clock counter for counting a clock between the peak and valley points and the counting is initiated and terminated in response to the detected peak and valley characteristics.

Processor 15 in step 518 calculates characteristics of the filtered SPO2 oximetric signal data including variation, variability, waveform density and average values. Processor 15 calculates coefficients representing reduction in blood flow volume from a patient heart to the particular anatomical location. The parameters in a calculation are adjusted and controlled via system adaptive control or user selection. In step 520 computation processor 15 uses the received filtered signal data in calculating a heart stroke volume (SV) of the patient comprising volume of blood transferred through the blood vessel in a heart cycle, in response to, a blood volume derived in response to oxygen content of patient blood and at least one factor representing reduction in blood flow volume from a patient heart to the particular anatomical location. Computation processor 15 also calculates CO. In step 526, mapping processor 22 uses predetermined mapping information associating ranges of calculated stroke volume or values derived from the calculated stroke volume with medical conditions and for mapping the calculated stroke volume to data indicating a medical condition of the patient. If processor 22 in step 526 determines a medical condition such as ventricular arrhythmia or related event indicating cardiac impairment or another abnormality is identified, processor 22 in step 535 uses the mapping information in generating an alert message identifying the medical condition and abnormality and communicates the message to a user and stores data indicating the identified condition and associated calculated parameters in repository 17. Processor 15 updates patient information and health status (such as in response to medication administration) which may affect SV calculation.

Processor 15 in step 523 adaptively adjusts the number of cycles in a calculation window used in SV calculation in step 520 and in SV averaging and adjusts a threshold employed to improve medical condition detection. If processor 22 in step 526 does not identify a medical condition or change in patient information or health status, processor 15 in step 529 determines patient medical and demographic data (age, weight, gender) and in step 531 calculates a CO/SV ratio and updates at least one factor representing reduction in blood flow volume from a patient heart to the particular anatomical location and repeats SV calculation in step 520 and steps 526, 529 and 531 until a medical condition is identified in step 526.

The SPO2 oximetric signal based non-invasive CO and SV characterization is used in different clinical applications, such as in an operating room (OR), intensive care unit (ICU) and critical care unit (CCU) and EM (emergency room) for monitoring patient health status. Deviation in CO and SV is determined in order to facilitate early detection of patient health abnormality including arrhythmias and pathology and to predict patient pathology and facilitate identification of suitable treatment.

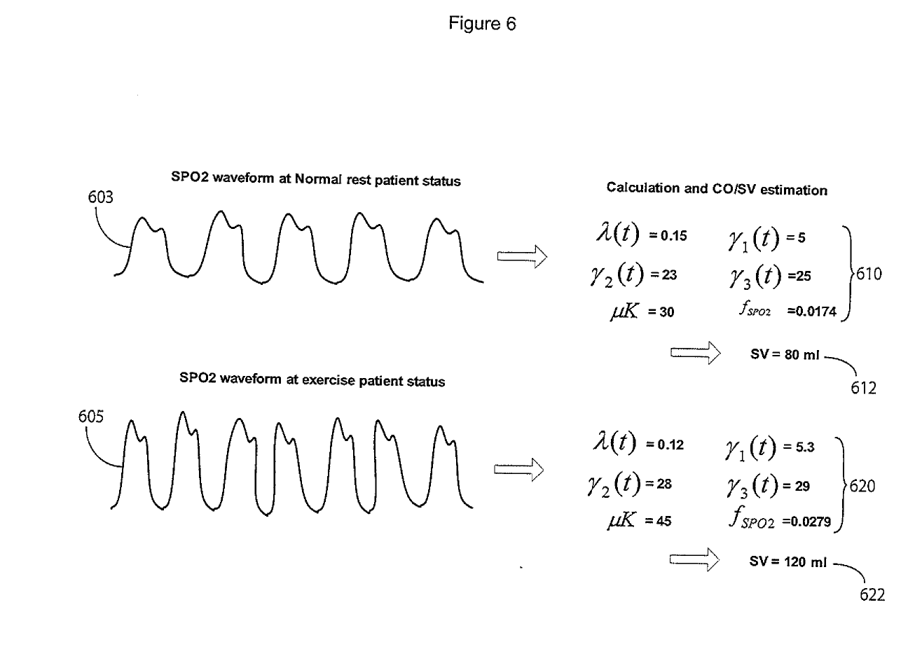

FIG. 6 illustrates SPO2 signal based CO and SV simulated calculation during a first normal rest episode and a second exercise episode of a patient. CO and SV are determined as previously described based on SPO2 signals 603 and 605 corresponding to the normal and exercise episodes, respectively. The determined CO and SV values of the two episodes are compared. The heart rate is 70 bpm (beats per minute) in the normal rest episode and 105 bpm during the exercise episode. Ratio coefficients and factors 610 are calculated in the rest episode as, γ₁ (t)=5, γ₂(t)=23, γ₃(t)=25, λ(t)=0.15 giving an SV value 612 of 80 ml. Ratio coefficients and factors 620 are calculated in the exercise episode as, γ₁(t)=5.3, γ₂(t)=28, γ₃(t)=29, λ(t)=0.12 giving an SV value 622 of 120 ml. It can be seen during exercise, the blood flow and SV value is higher than in rest since the human body and muscle needs more oxygen and blood (here system 10 selects window size for rest status as 10 cycles and 15 cycles for exercise status). The window size change helps to eliminate noise in the calculation caused by exercise, such as baseline changes. The SPO2 index ƒ_{SPO2} value is determined as previously described based on SPO2 waveform density, max, min, average. System 10 automatically compares parameters derived for the two different episodes. Different kinds of SPO2 waveform analysis are performed to facilitate determination of cardiac output and health status of a patient. Additionally, a threshold is set and adjusted to track cardiac function pathology. For example, by using a database of CO and SV values associated with different kinds of medical condition, a particular condition is identified for a specific patient heart output, e.g., a 20% threshold for patient CO changes based on SPO2 is used to determine abnormality of a monitored patient.

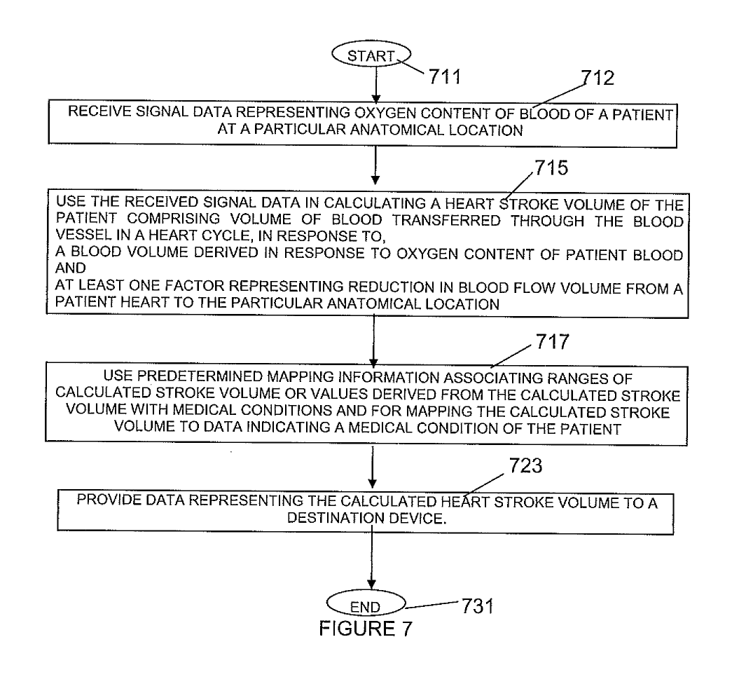

FIG. 7 shows a flowchart of a process used by system 10 (FIG. 1) for determining cardiac output or stroke volume. In step 712 following the start at step 711, input processor 12 receives signal data (e.g., digitally sampled data) such as a blood oxygen saturation (SPO2) signal and representing oxygen content of blood of a patient at a particular anatomical location. In step 715 computation processor 15 uses the received signal data in calculating a heart stroke volume of the patient comprising volume of blood transferred through the blood vessel in a heart cycle, in response to, a blood volume derived in response to oxygen content of patient blood and at least one factor representing reduction in blood flow volume from a patient heart to the particular anatomical location.

Computation processor 15 adaptively determines the at least one factor representing reduction in blood flow volume from a patient heart to the particular anatomical location in response to, an indicator indicating patient activity including at least one of rest and exercise, demographic characteristics of the patient comprising one or more of, age, height, weight, gender and pregnancy status and in response to at least one of, (a) heart rate, (b) respiration rate and (c) patient temperature. In one embodiment computation processor 15 determines the at least one factor representing reduction in blood flow volume from a patient heart to the particular anatomical location using an artificial neural network. The artificial neural network is configured using a training data set comprising data for the patient concerned or using a training data set selected from multiple training data sets of a population of patients sharing demographic data of the patient concerned, the demographic data comprising at least two of, age, height, weight, gender and pregnancy status.

In an embodiment, computation processor 15 determines the blood volume in response to a ratio between a blood volume in a vessel substantially at the particular anatomical location and oxygen content of the blood volume in the vessel and adaptively adjusts the determined blood volume in response to, (a) heart rate, (b) respiration rate, (c) patient temperature, (d) demographic characteristics of the patient and (e) an indicator indicating patient activity including at least one of rest and exercise Alternatively, computation processor 15 determines the blood volume in response to a density value calculated for the received signal data. The density value is calculated for the received signal data using a function of the form,

$$Density 1 = \frac{1}{N} {\int }_{i ∈ N} \vert {data}_{i} \vert d t$$

$$or Density 2 = \frac{1}{N} {\int }_{i ∈ N} {\vert {data}_{i} \vert}^{2} d t$$

where N is the number of data samples in the density calculation window, dataᵢ are data values in the received signal data.

In a further embodiment, computation processor 15 determines the blood volume derived in response to oxygen content of patient blood using at least one of (a) a Mean, (b) a Standard Deviation, (c) a Variation, (d) a Variability value of the received signal data and (e) a patient specific base value K. The computation processor adaptively adjusts K in response to at least one of; (a) patient demographic characteristics and (b) an indicator indicating patient activity including at least one of rest and exercise.

In step 717 mapping processor 22 uses predetermined mapping information associating ranges of calculated stroke volume or values derived from the calculated stroke volume with medical conditions and for mapping the calculated stroke volume to data indicating a medical condition of the patient. The predetermined mapping information associates ranges of the calculated stroke volume with particular patient demographic characteristics and with corresponding medical conditions and the system uses patient demographic data including at least one of, age weight, gender and height in comparing the calculated stroke volume with the ranges and generating an alert message indicating a potential medical condition. In step 723 output processor 20 provides data representing the calculated heart stroke volume and the indicated medical condition to a destination device. The process of FIG. 7 terminates at step 731.

A processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a computer, controller or microprocessor, for example, and is conditioned using executable instructions to perform special purpose functions not performed by a general purpose computer. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters, A user interface (UI), as used herein, comprises one or more display images, generated by a user interface processor and enabling user interaction with a processor or other device and associated data acquisition and processing functions.

The UI also includes an executable procedure or executable application. The executable procedure or executable application conditions the user interface processor to generate signals representing the UI display images. These signals are supplied to a display device which displays the image for viewing by the user. The executable procedure or executable application further receives signals from user input devices, such as a keyboard, mouth, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

The system and processes of FIGS. 1-7 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A system determines cardiac output and stroke volume by using non-invasive oximetric signals, such as blood oxygen saturation (SPO2) data to quantitatively determine blood flow. Further, the processes and applications may, in alternative embodiments, be located on one or more (e.g., distributed) processing devices on a network linking the units of FIG. 1. Any of the functions and steps provided in FIGS. 1-7 may be implemented in hardware, software or a combination of both.

What is claimed is:

1. A non-invasive system for determining cardiac output or stroke volume, comprising:
an input processor for receiving signal data representing oxygen content of blood of a patient at a particular anatomical location,
a computation processor for using the received signal data in calculating a heart stroke volume of said patient comprising volume of blood transferred through the blood vessel in a heart cycle, in response to,
a blood volume derived in response to oxygen content of patient blood and
at least one factor representing reduction in blood flow volume from a patient heart to said particular anatomical location; and
an output processor for providing data representing the calculated heart stroke volume to a destination device.

2. A system according to claim 1, wherein
the signal representing oxygen content of blood of said patient comprises a blood oxygen saturation (SPO2) signal.

3. A system according to claim 1, wherein
said signal data is digitally sampled data.

4. A system according to claim 1, including
a mapping processor for using predetermined mapping information associating ranges of calculated stroke volume or values derived from said calculated stroke volume with medical conditions and for mapping the calculated stroke volume to data indicating a medical condition of said patient and
said output processor provides data representing the indicated medical condition to a destination device.

5. A system according to claim 1, wherein
said computation processor determines said blood volume in response to a ratio between a blood volume in a vessel substantially at said particular anatomical location and oxygen content of said blood volume in said vessel.

6. A system according to claim 1, wherein
said computation processor determines said blood volume in response to a density value calculated for the received signal data.

7. A system according to claim 6, wherein
said density value is calculated for the received signal data using a function of the form,
$$Density 1 = \frac{1}{N} {\int }_{i ∈ N} \vert {data}_{i} \vert d t$$
$$or Density 2 = \frac{1}{N} {\int }_{i ∈ N} {\vert {data}_{i} \vert}^{2} d t$$
where N is the number of data samples in the density calculation window, dataᵢ are data values in the received signal data.

8. A system according to claim 1, wherein
said computation processor adaptively determines said at least one factor representing reduction in blood flow volume from a patient heart to said particular anatomical location in response to an indicator indicating patient activity including at least one of rest and exercise.

9. A system according to claim 1, wherein
said computation processor adaptively determines said at least one factor representing reduction in blood flow volume from a patient heart to said particular anatomical location in response to at least one of (a) heart rate, (b) respiration rate and (c) patient temperature.

10. A system according to claim 1, wherein
said computation processor adaptively determines said at least one factor representing reduction in blood flow volume from a patient heart to said particular anatomical location in response to demographic characteristics of said patient comprising at least two of, age, height, weight, gender and pregnancy status.

11. A system according to claim 1, wherein
said computation processor determines said blood volume in response to a ratio between a blood volume in a vessel substantially at said particular anatomical location and oxygen content of said blood volume in said vessel and adaptively adjusts the determined blood volume in response to an indicator indicating patient activity including at least one of rest and exercise.

12. A system according to claim 1, wherein
said computation processor determines said blood volume in response to a ratio between a blood volume in a vessel substantially at said particular anatomical location and oxygen content of said blood volume in said vessel and adaptively adjusts the determined blood volume in response to (a) heart rate, (b) respiration rate, (e) patient temperature and (d) demographic characteristics of said patient.

13. A system according to claim 1, wherein
said computation processor determines said at least one factor representing reduction in blood flow volume from a patient heart to said particular anatomical location using an artificial neural network.

14. A system according to claim 13, wherein
said artificial neural network is configured using a training data set comprising data for the patient concerned or using a training data set selected from a plurality of training data sets using demographic data of the patient concerned, said demographic data comprising at least two of, age, height, weight, gender and pregnancy status.

15. A system according to claim 1, wherein
said computation processor determines said blood volume derived in response to oxygen content of patient blood using at least one of (a) a Mean, (b) Standard Deviation, (c) a Variation and (d) a Variability value of the received signal data.

16. A system according to claim 1, including
a mapping processor for using predetermined mapping information associating ranges of calculated stroke volume or values derived from said calculated stroke volume with medical conditions and for mapping the calculated stroke volume to data indicating a medical condition of said patient and
said output processor provides data representing the indicated medical condition to a destination device wherein said predetermined mapping information associates ranges of the calculated stroke volume with particular patient demographic characteristics and with corresponding medical conditions and said system uses patient demographic data including at least one of, age weight, gender and height in comparing the calculated stroke volume with said ranges and generating an alert message indicating a potential medical condition.

17. A system according to claim 1, wherein
said computation processor determines said blood volume derived in response to oxygen content of patient blood in response to a patient specific base value K.

18. A system according to claim 17, wherein
said computation processor adaptively adjusts K in response to at least one of, (a) patient demographic characteristics and (b) an indicator indicating patient activity including at least one of rest and exercise.

19. A method for determining cardiac output or stroke volume, comprising the activities of:
receiving signal data representing oxygen content of blood of a patient at a particular anatomical location,
using the received signal data in calculating a heart stroke volume of said patient comprising volume of blood transferred through the blood vessel in a heart cycle, in response to,
a blood volume derived in response to oxygen content of patient blood and
at least one factor representing reduction in blood flow volume from a patient heart to said particular anatomical location; and
providing data representing the calculated heart stroke volume to a destination device.
